(19) United States
(12) Plant Patent Application Publication
(10) Pub. No.: US 2015/0143594 P1
(43) Pub. Date: May 21, 2015

(54) Corylus plant named 'Wepster'

(50) ;

(71) Applicants: Shawn A. Mehlenbacher, Corvallis, OR (US); David C. Smith, Corvallis, OR (US); Rebecca L. McCluskey, Corvallis, OR (US)

(72) Inventors: Shawn A. Mehlenbacher, Corvallis, OR (US); David C. Smith, Corvallis, OR (US); Rebecca L. McCluskey, Corvallis, OR (US)

(73) Assignees: University, Corvallis, OR (US); State of Oregon acting by and through the State Board of Higher Edu on behalf of Oregon State

(21) Appl. No.: 13/998,648

(22) Filed: Nov. 19, 2013

(51) Int. Cl. A01H 5/00

(52) U.S. Cl. PLT/152

(57) ABSTRACT

A new and distinct cultivar of Corylus plant named ‘Wepster’ characterized by high vigor and an upright-spreading plant habit, yellowish-green developing and fully expanded leaves during the spring and summer, resistance to eastern filbert blight caused by the fungus Anisogramma anomala (Peck) E. Muller, presence of random amplified polymorphic DNA markers 152-800 and 268-580 amplified by the polymerase chain reaction and which are linked to a dominant allele for resistance to eastern filbert blight from the cultivar Gasaway, expression of incompatibility alleles Si and S₂ in the styles, and DNA fingerprints at 8 of 20 microsatellite marker loci differ from both parents OSU 440.005 and ‘Tonda Pacifica’, and from one parent at an additional 12 marker loci.

### ACKNOWLEDGMENT OF GOVERNMENT SUPPORT

This invention was made with government support under Specific Cooperative Agreement No. 58-5358-9-447 awarded by the United States Department of Agriculture. The government has certain rights in the invention.

### BOTANICAL DENOMINATION

Corylus avellana

### VARIETY DESIGNATION

‘Wepster’

### BACKGROUND

The present Invention relates to a new and distinct cultivar of Corylus plant, botanically known as Corylus avellana, and hereinafter referred to by the name ‘Wepster’. Corylus avellana is in the family Betulaceae.

The new Corylus resulted from a controlled cross of female parent ‘Tonda Pacifica’ and male parent OSU 440.005 (unpatented) made in 1997 by Shawn A. Mehlenbacher and David C. Smith. ‘Tonda Pacifica’ is protected by U.S. Plant Pat. No. 22,715 (issued May 8, 2012). Hybrid seeds from the cross were harvested in August 1997, stratified, and seedlings grown in the greenhouse during the summer of 1998. From this cross, a total of 182 seedling trees were planted in the field in Corvallis, Oreg., USA in October, 1998. ‘Wepster’ was discovered and selected by the Inventors as a single plant within the progeny of the stated cross-pollination in a controlled environment in Corvallis, Oreg. It was originally assigned the designation OSU 894.030 (unpatented), which indicates the row and tree location of the original seedling. OSU 440.005 (unpatented) is from a cross of OSU 49.073×VR 8-32 (both unpatented). OSU 49.073 is from a cross of OSU 14.084×‘Tombul Ghiaghli’ (both unpatented). OSU 14.084 is from a cross of ‘Barcelona’×‘Daviana’ (both unpatented). VR 8-32 is from a cross of ‘Montebello’ ×‘Gasaway’ (both unpatented). ‘Tonda Gentile delle Langhe’ (unpatented), the female parent of ‘Tonda Pacifica’, is an important cultivar in Piemonte, northern Italy. ‘Barcelona’, Oregon's most widely planted hazelnut cultivar, is known as ‘Castanyera’ (unpatented) where it originated in Catalunya, Spain. ‘Daviana’, originally from England, is the most widely planted pollinizer in ‘Barcelona’ orchards. ‘Tombul Ghiaghli’, obtained from Greece, is similar to the Turkish cultivar ‘Mincane’ (unpatented). ‘Montebello’ is widely grown under several names in Sicily. ‘Gasaway’ is the source of a single dominant gene for resistance to eastern filbert blight.

The new cultivar was asexually reproduced by rooted suckers annually for eight years (2004- 05 and 2007-12) in Corvallis, Oreg. The new cultivar was also asexually propagated by whip grafting in 2005 in Corvallis, Oreg. The unique features of this new Corylus are stable and reproduced true-to-type in successive generations of asexual reproduction.

### SUMMARY

The following traits have been repeatedly observed and are determined to be the unique characteristics of ‘Wepster’. These characteristics in combination distinguish ‘Wepster’ as a new and distinct cultivar:

1. High vigor and upright-spreading plant habit.

2. Yellowish-green developing and fully expanded leaves during the spring and summer.

3. Resistance to eastern filbert blight caused by the fungus Anisogramma anomala (Peck) E. Muller.

4. Presence of random amplified polymorphic DNA markers 152-800 and 268-580 in DNA of ‘Wepster’ amplified by the polymerase chain reaction. These two markers are linked to a dominant allele for resistance to eastern filbert blight from the cultivar Gasaway (unpatented).

5. Expression of incompatibility alleles S₁ and S₂ in the styles.

6. DNA fingerprints at 8 of 20 microsatellite marker loci differ from both parents, ‘Tonda Pacifica’ and OSU 440.005, and from one parent at an additional 12 marker loci. Additional DNA fingerprints of grandparent ‘Tonda Gentile delle Langhe’, standard cultivar ‘Barcelona’, eastern filbert blight resistance source ‘Gasaway’ and five cultivars released by Oregon State University hazelnut breeding program are shown in attached Table 2.

In comparisons in two replicated trials conducted in Corvallis, Oreg., plants of the new Corylus differed from plants of the Corylus avellana cultivar ‘Barcelona’ (unpatented), and other cultivars and selections of Corylus avellana known to the Inventors primarily in nut size, nut shape, kernel percentage (ratio of kernel weight to nut weight), frequency of blank nuts (nuts lacking kernels), time of pollen shed, time of nut maturity, length of the husk or involucre, and plant size.

### BRIEF DESCRIPTION OF THE DRAWINGS

The accompanying colored photographs illustrate the overall appearance of the new cultivar, showing the colors as true as it is reasonably possible to obtain in colored reproductions of this type. Colors in the photographs may differ slightly from the color values cited in the detailed botanical description which accurately describe the colors of the new Corylus.

FIG. 1 shows typical nuts, raw kernels, and blanched kernels of ‘Wepster’ hazelnut (OSU 894.030) compared to those of ‘Yamhill’ hazelnut.

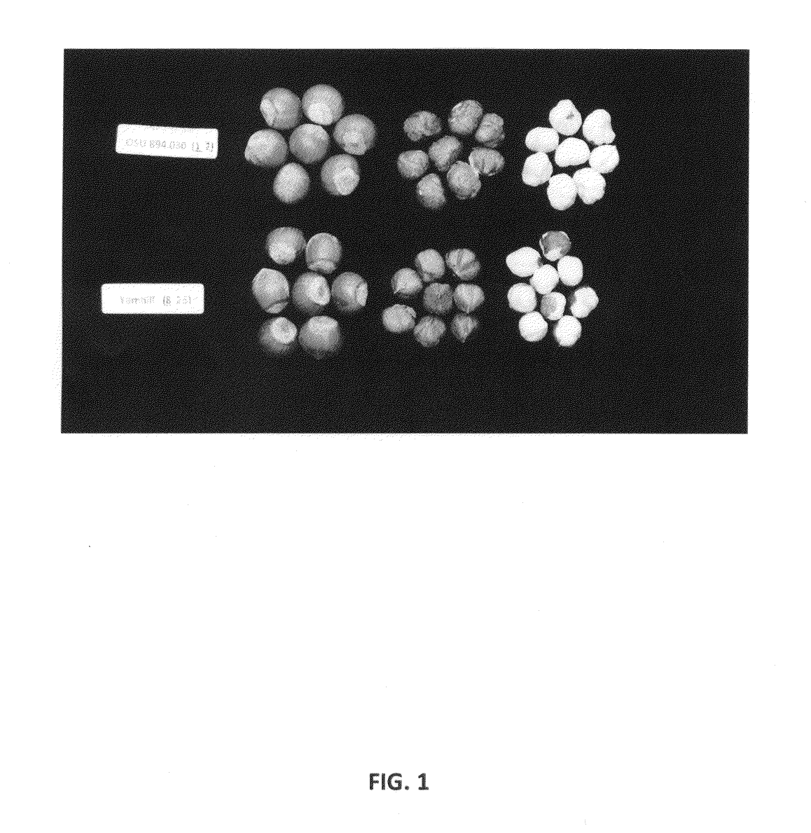

FIG. 2 shows the typical nuts, raw kernels, and blanched kernels of ‘Wepster’ hazelnut (OSU 894.030) compared to those of ‘Barcelona’, ‘Felix,’ and ‘York’ hazelnut cultivars.

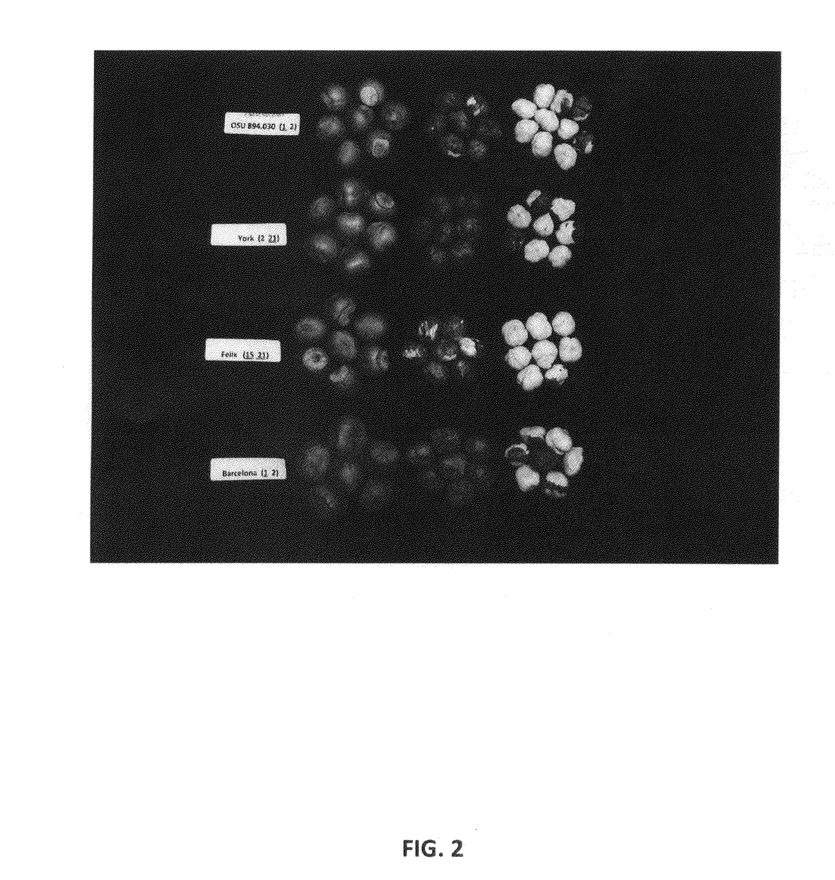

FIG. 3 shows the blanched kernels of ‘Wepster’ hazelnut (OSU 894.030).

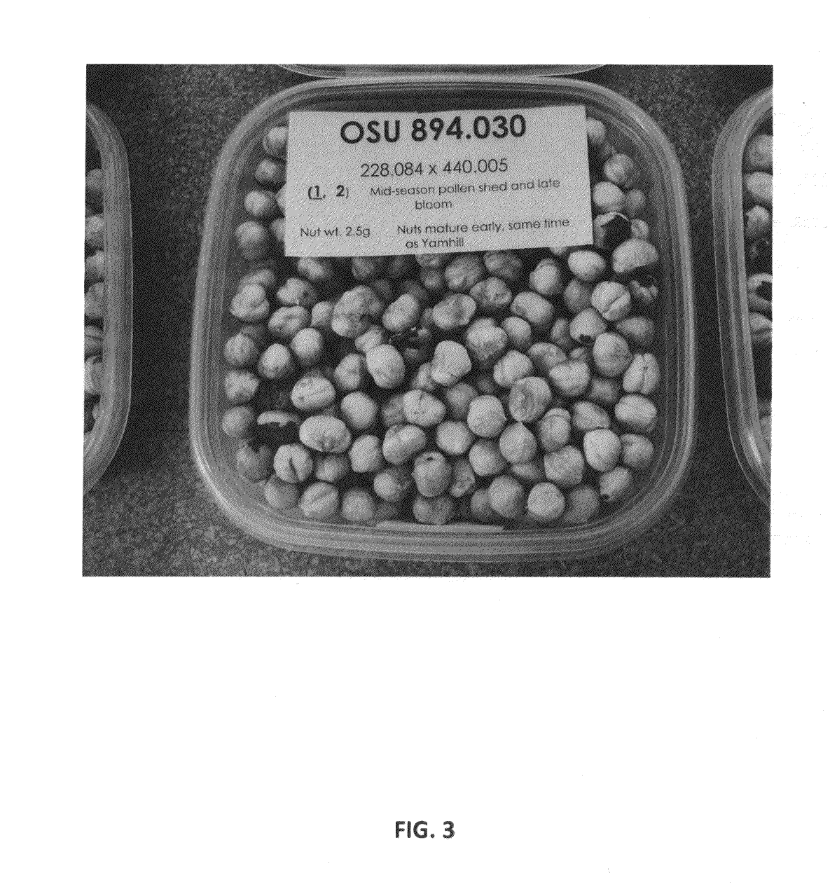

FIG. 4 shows the tree of the new cultivar ‘Wepster’ growing in a field in January, in Corvallis, Oreg.

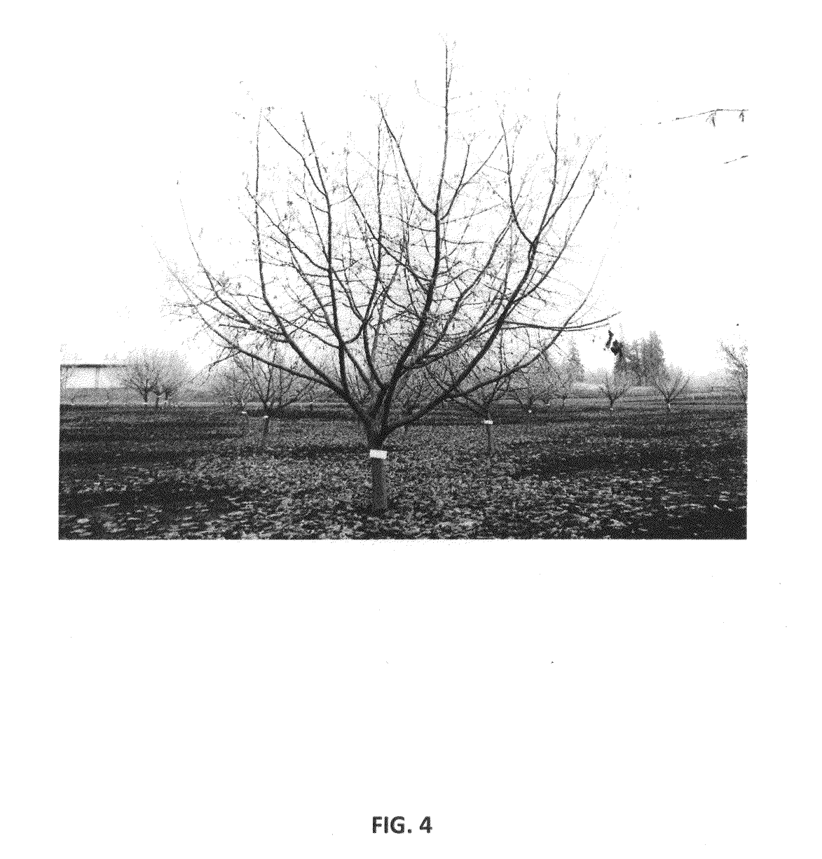

FIG. 5 shows a pruned tree of the new cultivar ‘Wepster’ in January, in Corvallis, Oreg.

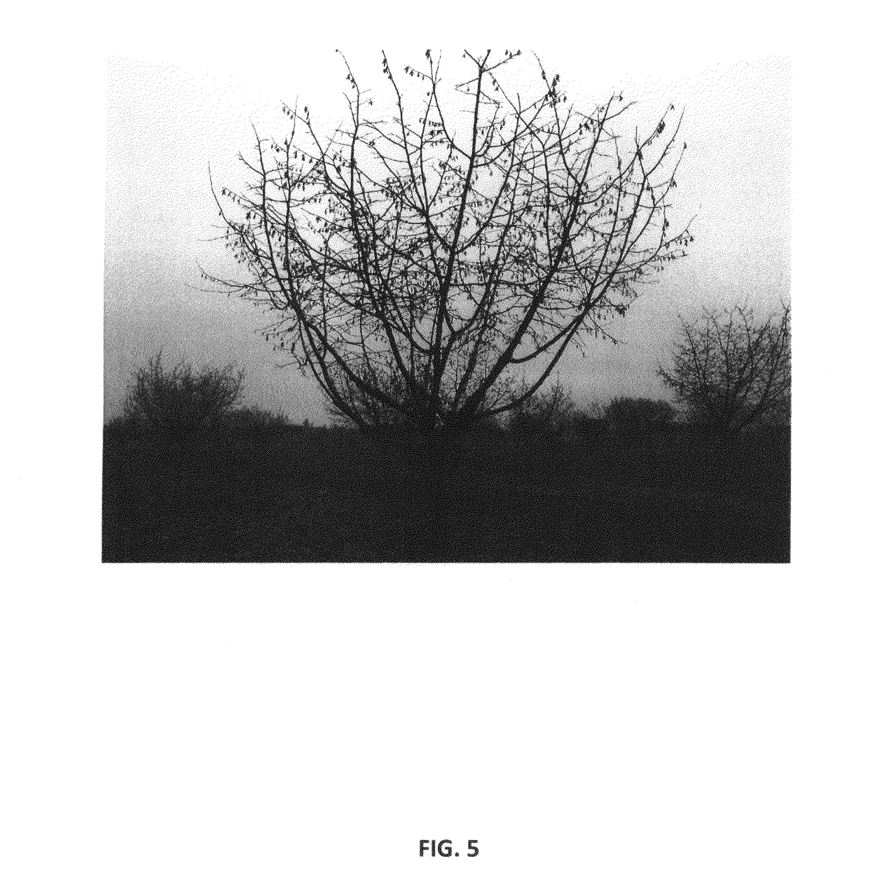

FIG. 6 shows the husks and nuts of ‘Wepster’ hazelnut.

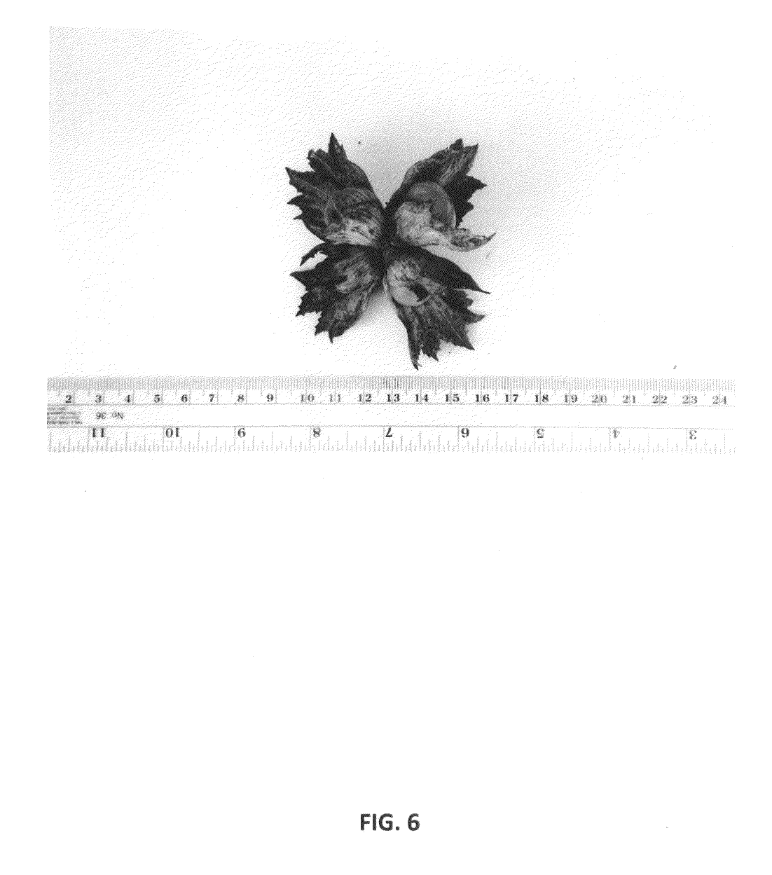

FIG. 7 shows a tree of the new cultivar ‘Wepster,’ 8^{th} leaf, growing in a field in the summer, in Corvallis, Oreg.

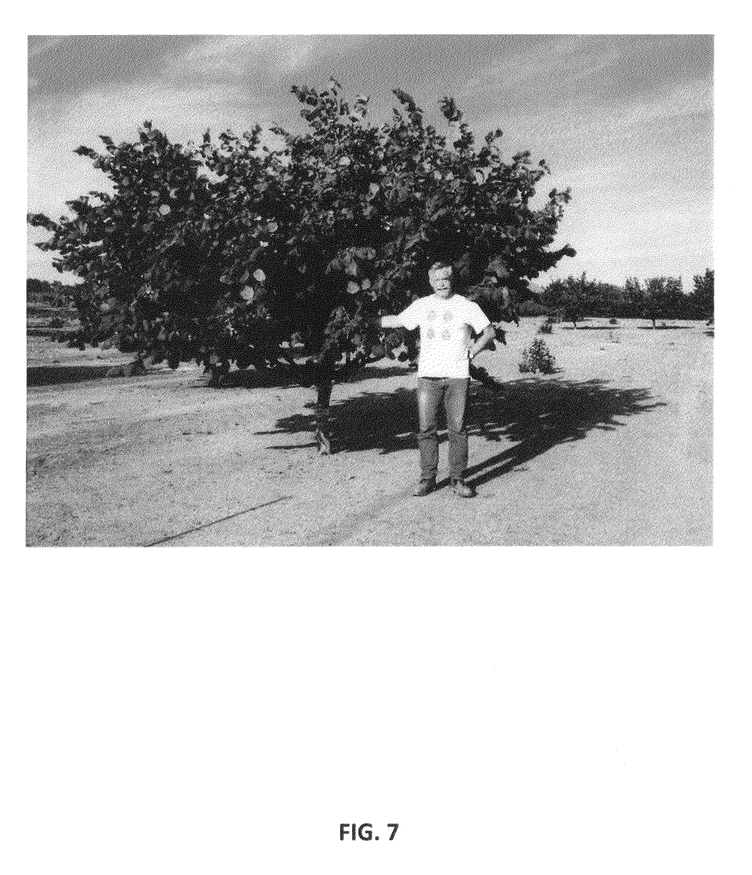

### DETAILED DESCRIPTION

The cultivar ‘Wepster’ has not been observed under all possible environmental conditions. The phenotype may vary somewhat with variations in environment such as temperature and light intensity, without, however, any variance in genotype. The aforementioned images and following observations and measurements describe plants grown in Corvallis, Oreg. under commercial practice outdoors in the field during the fall, winter and spring. Plants used for the photographs and description were propagated by tie-off layerage and growing on their own roots, and about seven years old. In the following description, color references are made to The Royal Horticultural Society Colour Chart, 1966 Edition, except where general terms of ordinary dictionary significance are used.
Botanical classification: Corylus avellana cultivar Wepster.
Parentage:
   Female, or seed, parent.—Corylus avellana cultivar Tonda Pacifica (U.S. Plant Pat. No. 22,715).
   Male, or pollen, parent.—Corylus avellana selection OSU 440.005 (unpatented).
Propagation (type rooted suckers):
   Time to initiate roots.—about 30 days at 20° C.
   Time to produce a rooted young plant.—about six months at 22° C.
   Root description.—Fine to thick; freely branching; creamy white in color.
Propagation (type whip grafting):
   Time to budbreak on the scions.—about 14 days at 25° C.
   Time to produce a grafted plant.—about six months at 25° C.
Plant description:
   Type.—perennial shrub. Upright-spreading plant habit.
   Growth and branching habit.—freely branching; about 15 lateral branches develop per plant. Pinching, i.e., removal of the terminal apices, enhances branching with lateral branches potentially forming at every node.
   Size.—plant height. — About 6 meters; plant diameter or spread is about 6 meters.
   Vigor.—high vigor growth habit.
Lateral branch description:
   Length.—about 32 cm.
   Diameter.—about 6 mm.
   Internode length.—about 3.0 cm.
   Texture.—smooth, glabrous.
   Strength.—strong.
   Color.—immature. — 152B; mature. — 152B.
Foliage description:
   Arrangement.—alternate, simple.
   Length.—about 10.2 cm.
   Width.—about 9.1 cm.
   Shape.—oblong to ovate.
   Apex.—obtuse to acute.
   Base.—cordate.
   Margin.—serrate.
   Texture, upper and lower surfaces.—slightly pubescent.
   Venation pattern.—pinnate.
Color — Developing foliage, upper surface 144A, lower surfaces: 187A.
   Fully expanded foliage, upper surface.—Spring and summer, 143A; late summer and fall, 143A.
   Fully expanded foliage, lower surface.—Spring and summer, 139C; late summer and fall, 139C.
   Venation, upper surface.—Spring and summer, 139C; late summer and fall, 139C.
   Venation, lower surface.—Spring and summer, 139D; late summer and fall, 139D.
Petiole description:
   Length.—about 2.7 cm.
   Diameter.—about 1.8 mm.
   Texture, upper and lower surfaces.—pubescent.
   Color.—upper surface: Spring and summer, 139D; late summer and fall, 139D. lower surface: Spring and summer, 139D; late summer and fall, 139D.
Flower description:
   Male inflorescences.—catkins, color prior to elongation 176C.
   Female inflorescence.—style color 048B.
Nut description:
   Length.—about 18.3 mm.
   Width.—about 19.0 mm.
   Depth.—about 16.6 mm.
   Nut shape.—round.
   Nut shape index [(width+depth)/2*length].—0.97.
   Nut compression index (width/depth).—1.15.
   Nut shell color.—164A.
   Nut weight.—about 2.39 grams.
   Kernel weight.—about 1.11 grams.
   Kernel percentage (kernel weight/nut weight).—about 43.9%.
Disease/pest resistance: Plants of the new Corylus are highly resistant to eastern filbert blight caused by the fungus Anisogramma anomala (Peck) E. Muller. Plants of the new Corylus are highly resistant to bud mites (Phytoptus avellanae Nal.), while plants of ‘Tonda Gentile delle Langhe’ are highly susceptible, and plants of ‘Barcelona’ are highly resistant.
Temperature tolerance: Plants of the new Corylus have been observed to tolerate temperatures from -10 to 38 degrees C. in the field in Corvallis, Oreg.

TABLE 1
Primers and annealing temperatures for the 20 microsatellite marker
loci used to fingerprint ‘Wepster’ and other hazelnut cultivars.

| | Repeat | | | | | | | | | Primers 5′-3′ | Primers 5′-3′ | |
|---|---|---|---|---|---|---|---|---|---|---|---|---|
| motif | Locus | Size | Tₐ | n | He | Ho | PIC | r | LG | (forward) | (reverse) | Reference |
| A614 | (TC)₁₇ | 125-156 | 60 | 14 | 0.85 | 0.85 | 0.84 | 0.00 | 6 | Hex-TGGCAGAG | R-GCAGTGGA | Gurcan et |
| | (CA)₁₀ | | | | | | | | | CTTTGTCAGCTT | GGATTGCTGACT | al. 2010 |
| | NNN(CA)₆ | | | | | | | | | (SEQ ID NO: 1) | (SEQ ID NO: 2) | |
| A616 | (AC)₁₁ | 136-162 | 60 | 13 | 0.85 | 0.85 | 0.83 | 0.00 | 8 | Fam-CACTCATAC | R-ATGGCTTTT | Gurcan et |
| | | | | | | | | | | CGCAAACTCCA | GCTTCGTTTTG | al. 2010 |
| | | | | | | | | | | (SEQ ID NO: 3) | (SEQ ID NO: 4) | |
| A640 | (CT)₁₅ | 354-378 | 67 | 11 | 0.80 | 0.73 | 0.77 | 0.04 | 10 | F-TGCCTCTGCAGTTA | Fam-CGCCATATAATT | Gurcan et |
| | (CA)₁₃ | | | | | | | | | GTCATCAAATGTAGG | GGATGCTTGTTG | al. 2010 |
| | | | | | | | | | | (SEQ ID NO: 5) | (SEQ ID NO: 6) | |
| B617 | (GA)₁₅ | 280-298 | 60 | 9 | 0.80 | 0.78 | 0.78 | 0.01 | 8 | Fam-TCCGTGTTGAGT | R-TGTTTTTGGTG | Gurcan et |
| | | | | | | | | | | ATGGACGA | GAGCGATG | al. 2010 |
| | | | | | | | | | | (SEQ ID NO: 7) | (SEQ ID NO: 8) | |
| B619 | (TC)₂₁ | 146-180 | 60 | 14 | 0.88 | 0.88 | 0.87 | 0.00 | 3 | Fam-AGTCGGCTCC | R-GCGATCTGA | Gurcan et |
| | | | | | | | | | | CCTTTTCTC | CCTCATTTTTG | al. 2010 |
| | | | | | | | | | | (SEQ ID NO: 9) | (SEQ ID NO: 10) | |
| B634 | (AG)₁₅ | 218-238 | 60 | 9 | 0.76 | 0.76 | 0.73 | 0.00 | 4 | Hex-CCTGCATCCA | R-GTGCAGAGG | Gurcan et |
| | | | | | | | | | | GGACTCATTA 60 | TTGCACTCAAA | al. 2010 |
| | | | | | | | | | | (SEQ ID NO: 11) | (SEQ ID NO: 12) | |
| B671 | (AG)₆NN | 221-249 | 60 | 13 | 0.86 | 0.88 | 0.84 | −0.01 | 9 | Hex-TTGCCAGTG | R-ACCAGCTCT | Gurcan et |
| | (GA)₁₇ | | | | | | | | | CATACTCTGATG | GGGCTTAACAC | al. 2010 |
| | | | | | | | | | | (SEQ ID NO: 13) | (SEQ ID NO: 14) | |
| B709 | (GA)₂₁ | 219-233 | 60 | 16 | 0.87 | 0.80 | 0.86 | 0.04 | 8 | Fam-CCACCAAC | R-GCGAAATGG | Gurcan et |
| | | | | | | | | | | TGTTTCACACCA | AGCTCTTGAAC | al. 2010 |
| | | | | | | | | | | (SEQ ID NO: 15) | (SEQ ID NO: 16) | |
| B733 | (TC)₁₅ | 161-183 | 60 | 8 | 0.68 | 0.68 | 0.63 | 0.00 | 7,2 | Ned-CACCCTCTT | R-CATCCCCTG | Gurcan et |
| | | | | | | | | | | CACCACCTCAT | TTGGAGTTTTC | al. 2010 |
| | | | | | | | | | | (SEQ ID NO: 17) | (SEQ ID NO: 18) | |
| B749 | (TC)₁₂ | 200-210 | 60 | 6 | 0.60 | 0.64 | 0.51 | −0.03 | 1 | Hex-GGCTGACA | R-TCGGCTAGG | Gurcan et |
| | | | | | | | | | | ACACAGCAGAAA | GTTAGGGTTTT | al. 2010 |
| | | | | | | | | | | (SEQ ID NO: 19) | (SEQ ID NO: 20) | |
| B767 | (TC)₁₅ | 198-238 | 60 | 16 | 0.87 | 0.80 | 0.86 | 0.04 | 8 | Fam-CCACCAACT | R-GCGAAATGG | Gurcan et |
| | (AT)₇ | | | | | | | | | GTTTCACACCA | AGCTCTTGAAC | al. 2010 |
| | | | | | | | | | | (SEQ ID NO: 21) | (SEQ ID NO: 22) | |
| B774 | (AG)₁₅ | 195-213 | 60 | 8 | 0.80 | 0.80 | 0.77 | 0.01 | 5 | Ned-GTTTTGCGA | R-TGTGTGTGGT | Gurcan et |
| | | | | | | | | | | GCTCATTGTCA | CTGTAGGCACT | al. 2010 |
| | | | | | | | | | | (SEQ ID NO: 23) | (SEQ ID NO: 24) | |
| B795 | (TC)₈N_{S} | 296-332 | 60 | 12 | 0.76 | 0.74 | 0.74 | 0.01 | NA | Fam-GACCCACAA | R-TGGGCATCAT | Gurcan et |
| | (CT)₇N_{S} | | | | | | | | | ACAATAACCTATCTC | CCAGGTCTA | al. 2010 |
| | (CT)₁₀ | | | | | | | | | (SEQ ID NO: 25) | (SEQ ID NO: 26) | |
| | N_{S}(TC)₅ | | | | | | | | | | | |
| C115 | (TAA)₅ | 167-226 | 60 | 14 | 0.80 | 0.80 | 0.77 | 0.00 | 4 | Fam-ATTTTCCGC | GTTTCCAGAT | Bassil et |
| | (GAA)₁₂ | | | | | | | | | AGATAATACAGG | CTGCCTCCATATAAT | al. 2005b, |
| | | | | | | | | | | (SEQ ID NO: 27) | (SEQ ID NO: 28) | Gokirmak et |
| | | | | | | | | | | | | al. 2009 |
| KG807 | (TAAA)AA | 226-248 | 54 | 4 | 0.67 | 0.78 | 0.60 | -0.07 | 11 | AAGCAAGAAAGGGA | FAM-CTTACAGATA | Gurcan and |
| | (TAAA)₂ | | | | | | | | | TGGT | AATGGCTCAAA | Mehlenbacher |
| | A(TAAA)₂ | | | | | | | | | (SEQ ID NO: 29) | (SEQ ID NO: 30) | 2010 |
| KG809 | (AGG)₆ | 333-345 | 55 | 5 | 0.66 | 0.64 | 0.60 | 0.01 | 4 | Hex-AGGCATCA | F-GGAAGGTGA | Gurcan and |
| | | | | | | | | | | GTTCATCCAA | GAGAAATCAAGT | Mehlenbacher |
| | | | | | | | | | | (SEQ ID NO: 31) | (SEQ ID NO: 32) | 2010 |
| KG811 | (GA)₁₇ | 240-278 | 58 | 12 | 0.83 | 0.82 | 0.81 | 0.01 | 2 | Ned-AAGGCGGCA | F-GAACAACTGAA | Gurcan and |
| | | | | | | | | | | CTCGCTCAC | GACAGCAAAG | Mehlenbacher |
| | | | | | | | | | | (SEQ ID NO: 33) | (SEQ ID NO: 34) | 2010 |
| KG827 | (CT)₁₃AA | 264-282 | 67 | 9 | 0.78 | 0.84 | 0.75 | −0.04 | 9 | Fam-AGAACTCCGAC | GAGGGAGCAAGTCAA | Gurcan and |
| | (CA)₇ | | | | | | | | | TAATAATCCTAACCC | AGTTGAGAAGAAA | Mehlenbacher |
| | | | | | | | | | | TTGC | (SEQ ID NO: 36) | 2010 |
| | | | | | | | | | | (SEQ ID NO: 35) | | |
| KG830 | (CT)₁₄ | 279-311 | 67 | 9 | 0.79 | 0.78 | 0.76 | 0.00 | 9 | Ned-TGGAGGAAGT | AAAGCAACTCATAGCT | Gurcan and |
| | GTATT | | | | | | | | | TTTGAATGGTAGTAG | GAAGTCCAATCA | Mehlenbacher |
| | (CA)₈ | | | | | | | | | AGGA | (SEQ ID NO: 38) | 2010 |
| | | | | | | | | | | (SEQ ID NO: 37) | | |
| Soman-G | (AAT)₅ | 193-200 | 54 | 3 | 0.60 | 0.98 | 0.51 | −0.27 | NA | Hex-TGGCGTTGCA | R-GCCATCTTTAG | unpub- |
| (= 856 − | | | | | | | | | | ACATATTCTC | AAAGTTCGATACAG | lished |
| MS1-13) | | | | | | | | | | (SEQ ID NO: 39) | (SEQ ID NO: 40) | |

Primer fluorescent tags are FAM, HEX, and NED.
Ta: annealing temperature (° C.)
N: number of alleles
He: expected heterozygosity
Ho: observed heterozygosity
PIC: polymorphism information content
r: estimated null allele frequency
LG: linkage group
Reference: for development and characterization

TABLE 2
Allele sizes in ‘Wepster’ and other hazelnut cultivars at 20 microsatellite loci.

| | | Tonda | OSU | Tonda Gentile | | | | | | | |
|---|---|---|---|---|---|---|---|---|---|---|---|
| Locus | Wepster | Pacifica | 440.005 | delle Langhe | Barcelona | Yamhill | Dorris | York | Felix | Santiam | Gasaway |
| A614 | 135/158 | 135/150 | 125/158 | 125/135 | 125/132 | 132/158 | 132/158 | 124/158 | 138/143 | 132/158 | 143/158 |
| A616 | 152/160 | 150/160 | 152/160 | 150/152 | 144/152 | 150/150 | 150/152 | 144/152 | 150/152 | 150/152 | 150/150 |
| A640 | 368/374 | 368/374 | 355/368 | 355/368 | 355/374 | 355/368 | 372/374 | 363/374 | 368/372 | 355/362 | 362/368 |
| B617 | 293/295 | 293/293 | 293/295 | 285/295 | 285/289 | 289/295 | 287/295 | 287/289 | 287/287 | 285/295 | 291/295 |
| B619 | 166/172 | 166/172 | 166/166 | 150/166 | 158/172 | 158/172 | 158/166 | 158/166 | 158/166 | 158/166 | 172/176 |
| B634 | 228/228 | 228/228 | 228/234 | 228/228 | 228/228 | 236/236 | 228/228 | 228/236 | 228/236 | 222/236 | 222/234 |
| B671 | 239/249 | 229/239 | 249/249 | 239/243 | 225/229 | 225/243 | 229/249 | 243/249 | 229/237 | 225/237 | 237/249 |
| B709 | 229/235 | 229/235 | 223/229 | 229/229 | 227/235 | 229/229 | 229/229 | 229/233 | 229/233 | 229/229 | 229/229 |
| B733 | 173/175 | 173/175 | 173/175 | 173/175 | 173/175 | 181/185 | 173/181 | 173/181 | 175/181 | 175/181 | 175/175 |
| B749 | 207/209 | 207/209 | 209/209 | 207/209 | 209/209 | 209/209 | 207/207 | 209/209 | 207/207 | 209/209 | 207/209 |
| B767 | 200/242 | 200/218 | 212/242 | 214/218 | 214/240 | 214/238 | 214/218 | 236/238 | 214/214 | 212/214 | 214/214 |
| B774 | 203/207 | 203/207 | 203/211 | 203/211 | 203/207 | 203/211 | 203/207 | 203/209 | 203/213 | 209/213 | 203/209 |
| B795 | 333/333 | 315/333 | 333/333 | 315/333 | 333/333 | 333/333 | 333/333 | 333/333 | 321/333 | 317/333 | 317/319 |
| C115 | 183/194 | 174/183 | 194/216 | 174/174 | 174/194 | 197/216 | 194/216 | 197/197 | 197/216 | 194/197 | 216/219 |
| KG807 | 252/252 | 228/252 | 252/252 | 238/252 | 238/252 | 230/252 | 242/252 | 242/252 | 238/242 | 242/252 | 242/252 |
| KG809 | 342/342 | 339/342 | 342/348 | 339/342 | 339/339 | 348/348 | 339/348 | 339/348 | 339/348 | 339/342 | 339/348 |
| KG811 | 257/257 | 245/257 | 257/267 | 257/267 | 261/267 | 251/261 | 257/267 | 257/257 | 251/267 | 257/267 | 257/261 |
| KG827 | 270/282 | 270/284 | 272/282 | 268/278 | 282/284 | 268/282 | 272/284 | 268/272 | 272/284 | 272/272 | 272/282 |
| KG830 | 295/305 | 291/295 | 295/305 | 291/295 | 291/295 | 291/295 | 295/297 | 295/295 | 293/303 | 291/295 | 291/305 |
| Soman-G | 196/200 | 196/200 | 196/196 | 196/200 | 196/200 | 196/200 | 196/200 | 196/200 | 196/200 | 196/200 | 196/196 |

### REFERENCES

Bassil N. V., Botta R., Mehlenbacher S. A. 2005a. Microsatellite markers in hazelnut: Isolation, characterization and cross-species amplification. J. Amer. Soc. Hort. Sci. 130:543-549.

Bassil N. V., Botta R., Mehlenbacher S. A. 2005b. Additional microsatellite markers of the European hazelnut. Acta Hort. 686:105-110.

Boccacci P., Akkak A., Bassil N. V., Mehlenbacher S. A., Botta R. 2005. Characterization and evaluation of microsatellite loci in European hazelnut (C. avellana) and their transferability to other Corylus species. Molec. Ecol. Notes 5:934-937.

Boccacci P., Akkak, A. and Botta, R. 2006. DNA typing and genetic relations among European hazelnut (Corylus avellana L.) cultivars using microsatellite markers. Genome 49:598-611.

Gökirmak T., Mehlenbacher S. A., Bassil N. V. 2009. Characterization of European hazelnut (Corylus avellana) cultivars using SSR markers. Genetic Resources and Crop Evolution 56:147-172.

Gürcan, K., S. A. Mehlenbacher and V. Erdogan. 2010a. Genetic diversity in hazelnut cultivars from Black Sea countries assessed using SSR markers. Plant Breeding (available on-line doi :10. 1111/j. 1439-0523. 2009. 01753. x).

Gürcan, K., S. A. Mehlenbacher, N. V. Bassil, P. Boccacci, A. Akkak and R. Botta. 2010b. New microsatellite markers for Corylus avellana from enriched libraries. Tree Genetics and Genomes (available on-line as DOI 10.1007/s11295-010-0269-y).

Gürcan, K. and S. A. Mehlenbacher. 2010. Development of microsatellite marker loci for European hazelnut (Corylus avellana L.) from ISSR fragments. Molecular Breeding (available on-line).

We claim:

1. A new and distinct cultivar of Corylus plant named ‘Wepster’, as illustrated and described.
